(19) United States
(12) Patent Application Publication
(10) Pub. No.: US 2024/0254536 A1
(43) Pub. Date: Aug. 1, 2024

(54) ANTIBIOTIC PIN ARRAY AND USES THEREOF

(71) Applicant: DrugArray, Inc., La Jolla, CA (US)

(72) Inventors: Dayu TENG, La Jolla, CA (US); Jieying LI, La Jolla, CA (US)

(21) Appl. No.: 18/561,437

(22) PCT Filed: May 17, 2022
(86) PCT No.: PCT/US2022/029652

§ 371 Date: Nov. 16, 2023

Related U.S. Application Data
(60) Provisional application No. 63/189,524, filed on May 17, 2021.

(51) Int. Cl. C12Q 1/18; C12M 1/00; C12M 1/22

(52) U.S. Cl. CPC C12Q 1/18; C12M 23/10; C12M 23/38

(57) ABSTRACT

Described herein are reagent loading devices configured to simultaneously deliver one or more test agents (e.g., antibiotics) to a sample such as a bacterial culture without having to individually deliver the test agents. These devices allow high throughput parallel processes without repetitive pipetting or liquid handling robotics. Also described herein are kits and systems for chemical or biological assays.

### CROSS-REFERENCE TO RELATED APPLICATION

This application claims priority to U.S. Provisional Patent Application Ser. No. 63/189,524, filed May 17, 2021, which is herein incorporated by reference in its entirety for all purposes.

### FIELD

Described herein are devices, kits, systems, and methods for efficiently screening antibiotic drugs without needing to individually dispense antibiotic drugs onto a bacteria culture, e.g., in a solid phase media.

### BACKGROUND

High-throughput screening allows researchers to quickly conduct a large number of chemical, biological, or pharmacological tests in parallel and is an important aspect of biological and chemical research—for example, in the development of new drugs. Such screening can be conducted manually or performed in an automated fashion using robotics or liquid handling devices to manipulate the samples of interest.

Kirby-Bauer disc diffusion susceptibility test has seen wide application in antibiotic drug screening, determining choices of antibiotics be used when treating an infection. This method relies on the inhibition of bacterial growth measured under standard conditions. See, for example, Bauer, A. W., W. M. M. Kirby, J. C. Sherris, and M. Turck. 1966. Am. J. Clin. Pathol. 36:493-496.

Efficiency of the standard Kirby-Bauer test equipment is generally low. In addition, manual dispensing of the drug discs is laborious, time-consuming and error-prone. Although dispensers for drug discs exist, they are generally expensive, take additional laboratory space, and still require manual loading of discs into the dispenser. Thus, more efficient and accurate methods of performing an antibiotic susceptibility test and similar tests for screening purposes are needed. The present disclosure addresses these and related needs.

### BRIEF SUMMARY

In some embodiments, disclosed herein are pin array devices, systems, and methods of use for a variety of applications. In some embodiments, in diagnostic labs, the pin array can be used to determine the susceptibility of bacteria isolated from a patient's infection to clinically approved antibiotics. This allows physicians to prescribe the most appropriate antibiotic treatment. In some embodiments, in drug discovery labs, especially bioprospecting labs, the pin array can be used to screen biological material (e.g., plant extracts, bacterial fermentation broths) and drug candidates (or combinations of drugs) for antibacterial activity. When bioprospecting, the pin array can be performed with paired strains of bacteria to achieve dereplication and provisionally identify antibacterial mechanism of action.

The pin array devices described herein allow a composition comprising an agent (e.g. an antibiotic drug) to be simultaneously applied to target cells (e.g. bacteria cultured on LB agar dish). This is achieved by the devices having a plurality of first pins that may be loaded with one or more compositions comprising an agent. As such, the tip of the first pins may each be loaded with a composition comprising an agent corresponding to the desired delivery configuration to the plurality of types and amounts of compositions and/or agents. It should be appreciated that in some variations of the pin array devices described herein, the orientation of the pin array may be marked with a second pin (orientation pin), such that the exact configuration of the composition and/or agents loaded onto the tips of the first pins may be readily known.

Together, these devices allow high throughput parallel processes without repetitive pipetting or liquid handling robotics. Also described herein are kits for chemical or biological assays, as well as methods for using the pin array devices described herein.

Generally, the pin array devices described herein may comprise a plurality of first pins and a plate having a proximal surface and a distal surface, wherein the plurality of first pins are integral to the plate and are on the distal surface of the plate, wherein the plurality of first pins form an array, and optionally wherein the array comprising first pins is in contact with a target cell culture (e.g. a bacteria lawn) when performing an assay. In some of these variations, the device further comprises a second pin. In some of these variations, the second pin is capable of marking the orientation of the array. In some of these variations, the second pin is outside of the array. In some variations, the plurality of first pins may comprise at least about 16 pins. In some variations, the plurality of first pins may comprise at least about 25 pins. In some variations, the plurality of first pins may comprise at least about 36 pins. In some variations, the plurality of first pins may comprise at least about 49 pins. In some variations, the plurality of first pins may comprise at least about 64 pins. In some variations, the plurality of first pins may comprise at least about 81 pins. In some variations, the plurality of first pins may comprise at least about 100 pins. In some variations, the pin array devices described herein may comprise a plurality of first pins, a second pin, a plate having a proximal surface and a distal surface, wherein the plurality of first pins and the second pin are integral to the plate and are on the distal surface of the plate, wherein the plurality of first pins form an array, wherein one or more compositions comprising an agent is pre-loaded onto the first pins and/or the second pin, wherein the second pin is capable of marking the orientation of the array and optionally the second pin is outside of the array.

In some of the variations, the plate is round and comprises a first rim on the distal surface of the plate. In some variations, the device further comprises a protective dish, wherein the dish comprises a bottom plate and a second rim disposed on the bottom plate, wherein the bottom plate of the dish comprises a proximal surface and a distal surface. In some variations, the plate is capable of fittingly engage the protective dish, and wherein the plate and the dish do not rotate relative to each other when a tight fit is formed.

In some variations of the pin array devices described herein, the length of the first pins is shorter than the distance between the distal surface of the plate and the proximal surface of the bottom plate when the plate and the protective dish fittingly engage each other, thereby preventing the tips of the first pins from touching the bottom plate. In some embodiments, the second pin is longer than the first pins, or the device further comprises a third pin on the proximal surface of the plate that is longer than the first pins. In some embodiments, the length of the second pin and/or the length of the third pin is about the same as the distance between the distal surface of the plate and the proximal surface of the bottom plate when the plate and the protective dish fittingly engage each other, thereby preventing the tips of the first pins from touching the bottom plate.

In some variations of the pin array devices described herein, the first pins are preloaded with one or more compositions comprising an agent. In some variations, each of the first pins is loaded with the same composition comprising an agent at various amounts. In some variations, each of the first pins is loaded with a different composition comprising an agent. In some variations, some of the first pins are loaded with the same composition comprising an agent at various amounts. In some embodiments, some of the first pins are loaded with a different composition comprising an agent. In some variations, the composition comprises one or more antibiotic drugs. In some variations, the one or more compositions comprising an agent are in the form of a powder, a solution, an emulsion, a film, or a gel.

In some variations of the pin array devices described herein, the length of the closed tips of the first pins is about 1 mm to about 50 mm. In some embodiments, any or more of the tips have a length between about 2 mm and about 30 mm. In some embodiments, any or more of the tips have a length of about 2.5 mm, about 5 mm, about 7.5 mm, about 10 mm, about 12.5 mm, about 15 mm, about 17.5 mm, about 20 mm, about 22.5 mm, about 25 mm, about 27.5 mm, about 30 mm, or in a range between any of the aforementioned values. In some embodiments, any two or more of the first pins have tips of the same or substantially the same length. In some embodiments, all of the first pins have tips of the same or substantially the same length. In some embodiments, two first pins have tips of substantially the same length when one tip has a length that is no more than 1%, no more than 2%, no more than 5%, or no more than 10% longer or shorter than that of the other tip. In some embodiments, any two or more of the first pins may have tips of different lengths.

In some variations, the largest cross-section diameter of the closed tips of the first pins is about 0.1 mm to about 10 mm. In some embodiments, any or more of the tips have a cross-section diameter between about 0.5 mm and about 8 mm. In some embodiments, any or more of the tips have a cross-section diameter of about 0.15 mm, about 0.25 mm, about 0.5 mm, about 0.75 mm, about 1 mm, about 1.25 mm, about 1.5 mm, about 1.75 mm, about 2 mm, about 2.25 mm, about 2.5 mm, about 2.75 mm, about 3 mm, about 3.5 mm, about 3.75 mm, about 4 mm, about 4.5 mm, about 4.75 mm, about 5 mm, about 5.5 mm, about 5.75 mm, about 6 mm, about 6.5 mm, about 6.75 mm, about 7 mm, about 7.5 mm, about 7.75 mm, about 8 mm, or in a range between any of the aforementioned values. In some embodiments, any two or more of the first pins have tips of the same or substantially the same cross-section diameter. In some embodiments, all of the first pins have tips of the same or substantially the same cross-section diameter. In some embodiments, two first pins have tips of substantially the same cross-section diameter when one tip has a cross-section diameter that is no more than 1%, no more than 2%, no more than 5%, or no more than 10% greater or less than that of the other tip. In some embodiments, any two or more of the first pins may have tips of different cross-section diameters.

In some variations, the distal end of the closed tips is concave. In some variations, the depth of concaved distal end is about 1 sm to about 5 mm.

Also described herein are kits for a chemical or biological assay according to any one of the variations described herein. In some variations, the kit further comprises one or more containers (e.g. petri dishes) capable of fittingly engage the device, and wherein the one or more containers and the device do not rotate relative to each other when a tight fit is formed. In some variations, the containers (e.g. petri dishes) contains one or more culture media, and optionally wherein the culture media is a solid phase media. In some variations, the kit further comprises one or more compositions comprising an agent, and wherein the first pins and/or the second pin can be loaded with one or more compositions in the kit.

Also described herein are methods for using the pin array devices described herein. Generally, the methods may comprise coupling the pin array devices loaded with one or more compositions comprising an agent to a target cell culture. The effect of the compositions and/or agents can be assessed from the reactions of the target cells upon contacting the compositions and/or agents.

In some variations, the methods may comprise i) preparing a target cell culture (e.g. a bacteria) on a solid culture media in a dish; ii) applying one or more compositions comprising an agent (e.g. an antibiotic drug) to tips of a plurality of first pins, wherein the first pins are integral to a plate that comprises a proximal surface, a distal surface and a second pin, wherein the first pins are on the distal surface of the plate, wherein the plurality of first pins form an array, wherein the second pin marks the orientation of the array and optionally the second pin is outside of the array; and iii) iii) stamping the tips of the plurality of first pins to the target cell culture, wherein the effects of the reagents on the target cell culture are analyzed.

In some variations, the methods may comprise i) preparing a target cell culture (e.g. a bacteria) on a solid culture media in a dish; ii) stamping the tips of a plurality of first pins pre-loaded with one or more compositions comprising an agent (e.g. an antibiotic drug) to the target cell culture, wherein the first pins are integral to a plate that comprises a proximal surface and a distal surface, wherein the first pins are on the distal surface of the plate, and wherein the plurality of first pins form an array, and iii) analyzing the effects of the reagents on the target cell culture.

In some of these variations of the methods described herein, the target cell culture comprises a bacteria and the compositions comprise one or more antibiotic drugs, and wherein the effects of the antibiotic drugs are analyzed by comparing sizes of inhibition zones around the tips of the first pins. In some embodiments, the effects of the agents are analyzed while the plurality of first pins are in contact with the target cell culture. In some embodiments, the effects of the agents are analyzed after the plurality of the first pins are separated from the target cell culture. In some embodiments, the plurality of the first pins are in contact with the target cell culture for at least about 30 minutes before the effects of the agents on the target cell culture are analyzed.

### BRIEF DESCRIPTION OF THE DRAWINGS

FIG. 1 is a schematic overview of the Kirby-Bauer susceptibility test.

FIG. 2 is a perspective view of a plate, a plurality of first pins, a second pin, and a first rim, respectively.

FIG. 3 is a perspective view of a plate comprising a first rim, a plurality of first pins, a second pin, and a protective dish comprising a second rim.

FIGS. 4A-4H are perspective views of tips of first pins of a pin array device.

FIG. 5A is an exemplary assay by a 7×7 pin array device loaded with various amounts of dried (e.g., lyophilized) ampicillin on a bacteria culture on an LB agar plate.

FIG. 5B is a schematic representation of the experimental configuration of FIG. 4A.

FIG. 6A is an exemplary bacteria susceptibility assay by a 7×7 pin array device loaded with various amounts of dried ampicillin on a bacteria culture on an LB agar plate.

FIG. 6B is a schematic representation of the experimental configuration of FIG. 5A.

FIGS. 7A-7B show bacteria susceptibility assays on LB agar media after overnight storage at 4° C. and in Styrofoam box with ice packs, respectively.

FIGS. 8A-8B show bacteria susceptibility assays on LB agar media on the same day and after storage at 4° C. for 5 weeks, respectively.

FIGS. 9A-9B show exemplary arrays formed by the closed tips of an exemplary pin array device.

FIG. 10 illustrates an example of a computing device 1000, in accordance with an embodiment. In some embodiments, the computing device is configured to be in the disclosed systems and is configured to perform the operational methods associated with the systems disclosed herein.

FIGS. 11A-11B show bacteria susceptibility assays on LB agar media using various combinations of antibiotics.

FIGS. 12A-12B show examples of the orientation pin relative to the first pins.

### DETAILED DESCRIPTION

The disk diffusion test (also known as the agar diffusion test, Kirby-Bauer test, disc-diffusion antibiotic susceptibility test, disc-diffusion antibiotic sensitivity test and KB test) is a culture-based microbiology assay used in diagnostic and drug discovery laboratories. In diagnostic laboratories, the test is performed by inoculating the surface of an agar plate with bacteria isolated from a patient's infection. Antibiotic-containing paper disks are then applied to the agar and the plate is incubated. If an antibiotic stops the bacteria from growing or kills the bacteria, there will be an area around the disk where the bacteria have not grown enough to be visible. This is called a zone of inhibition. The susceptibility of the bacterial isolate to each antibiotic can then be semi-quantified by comparing the size of these zones of inhibition to databases of information on known antibiotic-susceptible, moderately susceptible and resistant bacteria. In this way, it is possible to identify the most appropriate antibiotic for treating a patient's infection. Although the disk diffusion test cannot be used to differentiate bacteriostatic and bactericidal activity, it is less cumbersome than other susceptibility test methods such as broth dilution.

In drug discovery labs, the disk diffusion test may be performed slightly differently than in diagnostic labs. In this setting, it is not the bacterial strain that must be characterized, but a test substance (e.g., a plant or microbial extract, or an antibiotic or antibiotic combination). The agar plate is therefore inoculated with a bacterial strain of known phenotype, and disks containing the test substance are applied to the surface.

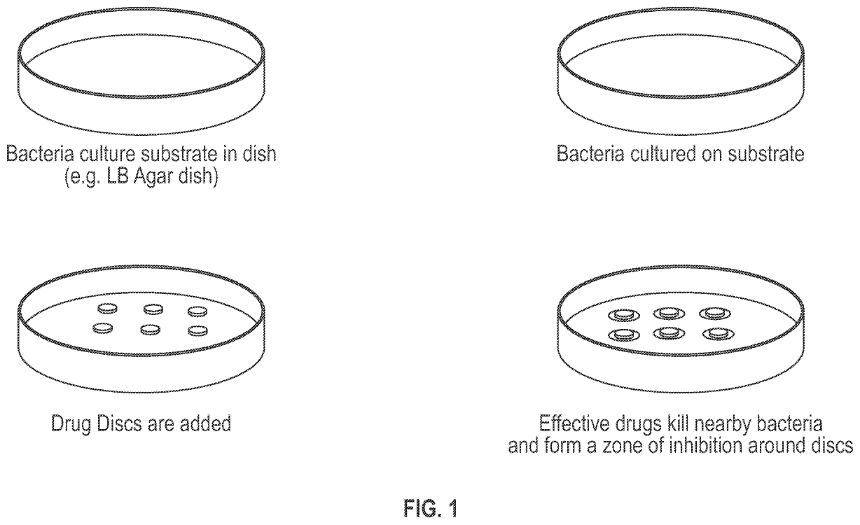

When performing a Kirby-Bauer disc diffusion test, an agent—for example, an antibiotic drug—is often impregnated into a filter paper disc and placed onto a bacteria culture, e.g. onto the surface of bacteria culture in an LB agar dish. A schematic representation of the experimental procedure of Kirby-Bauer susceptibility test is shown in FIG. 1, wherein after addition of drug discs, the drugs will kill nearby bacteria and zones of inhibition are formed around the drug discs. However, efficiency of the standard Kirby-Bauer test equipment is generally low, as the drug discs are commonly 6-6.5 mm in diameter, limiting the number of discs per standard 10 cm petri dish to 4-8. Manual dispensing of the drug discs is laborious, time-consuming and error-prone. Although dispensers for drug discs exist, they are generally expensive, take additional laboratory space, and still require manual loading of discs into the dispenser.

The pin array devices described herein allow a composition comprising an agent (e.g. an antibiotic drug) to be simultaneously applied to target cells (e.g. bacteria cultured on LB agar dish). This is achieved by the devices having a plurality of first pins that may be loaded with one or more compositions comprising an agent. As such, the tip of the first pins may each be loaded with a composition comprising an agent corresponding to the desired delivery configuration to the plurality of types and amounts of compositions and/or agents. For example, zone of inhibition can be formed around the distal tip of each pin and the size of the zone can be used as a qualitative, semi-quantitative, or quantitative measure of antibacterial potency. In some embodiments, zone of inhibition sizes can be used for the purpose of dereplication. This can be achieved by testing each sample against paired strains of bacteria (e.g. streptomycin-susceptible and -resistant strains to identify streptomycin-containing samples). Paired strains (e.g. wild type and target overexpressing strains) can also be used to identify antibacterial mechanism of action.

This device allow high throughput parallel processes without repetitive pipetting or liquid handling robotics. It should be appreciated that in some variations of the pin array devices described herein, the orientation of the pin array may be marked with a second pin (orientation pin), such that the exact configuration of the composition and/or agents loaded onto the tips of the first pins may be readily known. Also described herein are kits for chemical or biological assays, as well as methods for using the pin array devices described herein.

### I. Pin Array Device

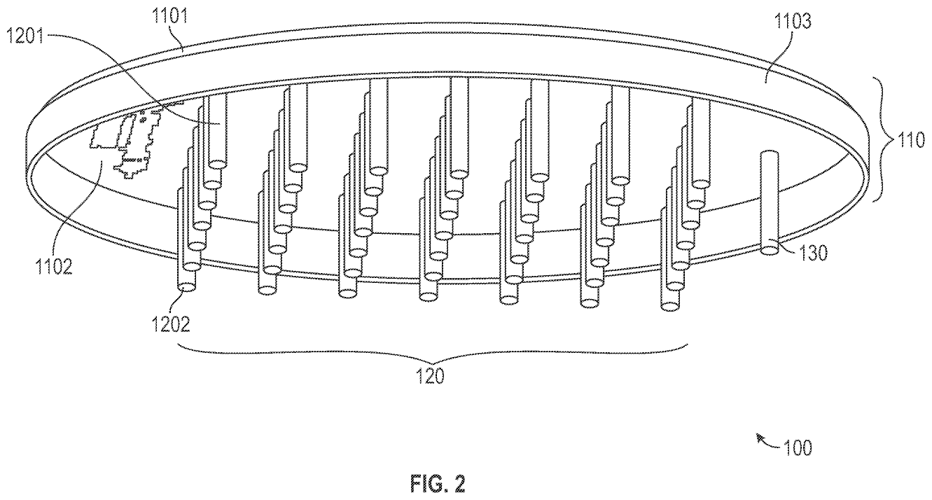

FIG. 2 illustrates a perspective view of one embodiment of the pin array device 100. In general, the pin array device 100 may comprise a plate 110 comprising a proximal surface 1101 and a distal surface 1102, a plurality of first pins 120, and a second pin 130, wherein the plurality of first pins and the second pin are integral to the distal surface of the plate 1102. A composition comprising an agent (e.g. an antibiotic drug) may be loaded onto the tips of the plurality of first pins 1202, as described in more detail below. In some variations, the pin array device may be used by coupling the first pins to a target cell culture, e.g., in solid phase (e.g. a bacteria culture on the surface of an LB agar media), such that the first pins contact the target cells at or beneath the surface of the target cell culture.

a. Plate

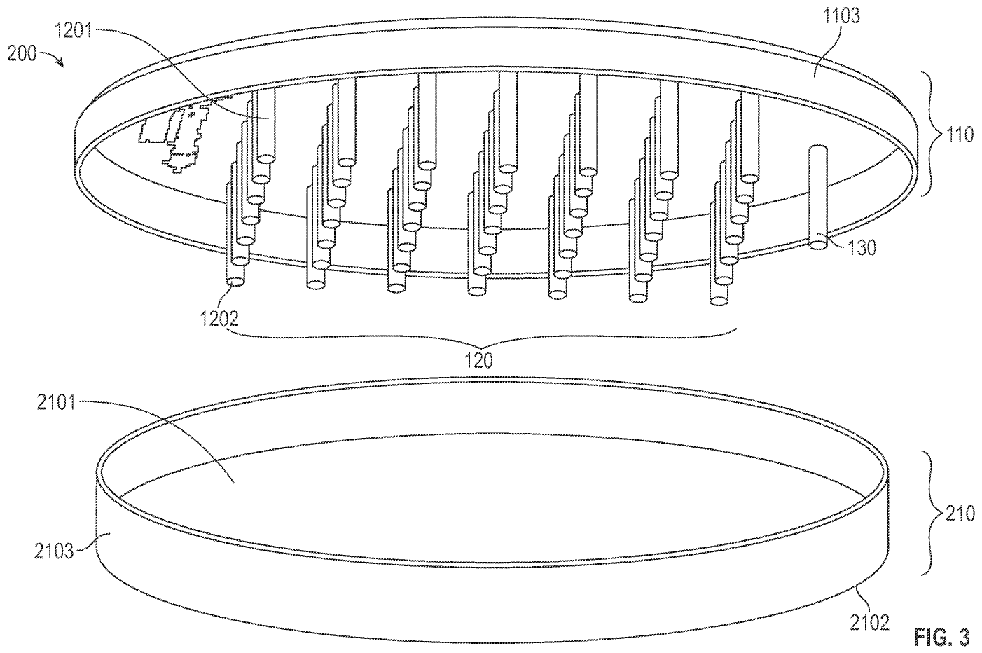

The plate 110 may form the top portion of the pin array device. In the variation shown in FIGS. 2 and 3, the plate may comprise a first rim 1103. In some variations, the first rim may be on the distal surface of the plate 1102, while in other variations, the first rim may be attached in any suitable manner (e.g. using adhesives such as glues or other adhesive polymers, welding, mechanical fasteners, chemical bonding, or a combination of these methods).

In some variations, the plate (e.g., dish) 110 is round. It should be appreciated, however, that the plate 110 need not be round, and furthermore, it need not comprise the first rim 1103. In some variations, for example, the plate 110 may define any polygon (e.g. a triangle, quadrilateral (e.g. parallelogram, trapezoid), pentagon, hexagon, etc.). In some variations, the plate is square. In some embodiments, the plate is rectangular. In some variations, the plate is or comprises a lid configured to cover a container such as a dish, where microorganisms can be provided on a surface in the container.

b. First Pins

In the variations shown in FIGS. 2 and 3, the pin array devices 100 and 200, respectively, may comprise a plurality of first pins 120. Each of the first pins 120 may comprise a stem 1201 and a tip 1202, respectively, described in more detail below. The first pins may comprise any suitable material or materials, such as but not limited to plastic, silicon, metal, or a polymer. In some variations the stems and closed tips may comprise the same materials, while in other variations they may comprise different materials. In some embodiments, the tips can comprise a hydrogel, a porous material (e.g., as a filling inside a packet at the end of a tip), an absorbent material (e.g., as a filling inside a packet at the end of a tip), or any combination thereof.

In variations in which the first pins are configured to be used to contact the target cells, the length of the first pins may be such that when the reagent loading device is coupled to a target cell culture, the tips of the first pins 1202 may be contacting the target cells at the surface of the target cell culture. In other variations, the tips of the first pins 1202 may be fully submerged beneath the surface of the target cell culture. In some variations, the length of the first pins may be about 1 mm to about 2 mm, about 2 mm to about 4 mm, about 4 mm to about 6 mm, about 6 mm to about 8 mm, about 8 mm to about 1 cm, about 1 cm to about 2 cm, about 2 cm to about 4 cm, about 4 cm to about 6 cm, longer than about 6 cm, about 1 mm to about 6 cm, about 1 mm to about 1 cm, about 2 mm to about 1 cm, about 2 mm to about 1.5 cm, about 2 mm to about 2 cm, about 2 mm to about 2.5 cm, about 2 mm to about 3 cm, or about 1 cm to about 6 cm. It should be appreciated that each of the first pins need not have the same configuration, for instance, any two or more of the first pins can be different lengths.

In some embodiments, the first pins are configured such that at least one first pin, upon the pin array engaging the bottom plate (e.g., 2101), can be partially inserted into a target cell culture. In some embodiments, the first pins are configured such that at least one first pin, upon the pin array engaging the bottom plate (e.g., 2101), is not inserted into a target cell culture. In some embodiments, the reagent at the distal tip of the first pin can touch the target cell culture. In some embodiments, the distal tip of the first pin can touch the target cell culture.

As mentioned above, the first pins (e.g. first pins 120 of the pin array device 100 or 200) may comprise tips (e.g. tips 1202 of pins 120 of the pin array device 100 or 200), which may each be configured to be loaded with a composition comprising an agent (e.g. an antibiotic drug). In some embodiments, the pins are pre-loaded with one or more compositions comprising an agent on the tips. It should be appreciated that each of tips of the first pins need not be pre-loaded with one or more compositions comprising an agent. It should also be appreciated that each of the tips of the first pins need not be loaded with the same composition and/or agent.

In some embodiments, the tips may be closed tips. The closed tips may be “closed” in the sense that they may not comprise an opening at the distal end that is connected to a cavity in the stem of the first pins through which the composition travels when it is deposited by the closed tip. This is in contrast to a device such as a pipette or the like, which comprises a cavity in the stem within which a composition is held, and an opening out of the cavity through which the composition travels when it is deposited by the pipette. A pipette or the like generally holds a composition within a cavity at least in part due to a partial vacuum within the cavity. In contrast, the closed tips of pin array device may be designed to hold the reagent outside the tip (not within a cavity in the stem) due to the interactions (e.g., adhesive forces) between the closed tip and the composition and due to interactions (e.g., surface tension) within the composition.

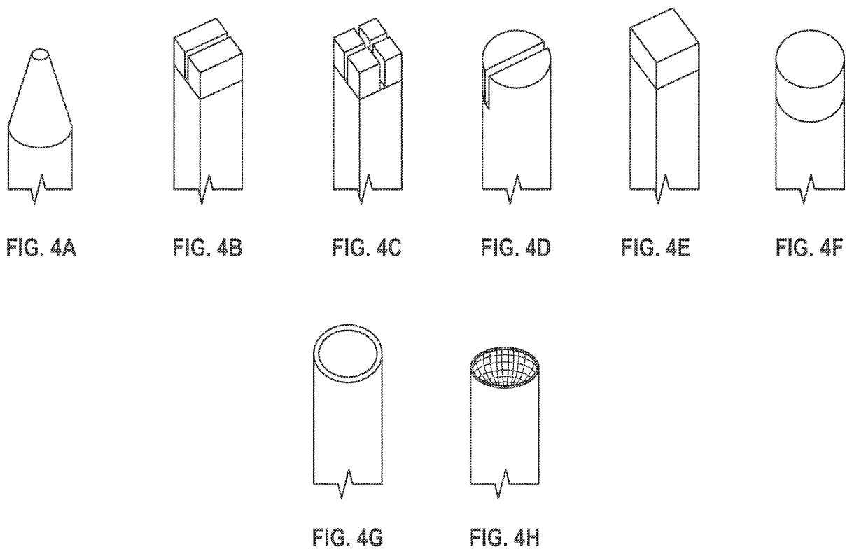

The closed tips (e.g., closed tips 1202) may have any suitable geometry for holding the reagent in such a way, including but not limited to a pointed shape or cone having a blunt tip (see FIG. 4A), square shape (see FIGS. 4B-4C and 4E), circular shape (see FIGS. 4D and 4F-4H), or the like. In some variations, the closed tips may be flat (see FIGS. 2-3 and FIG. 4F). In other variations, the closed tips may comprise a depression—for example, they may be concave (e.g., have a hemispherical depression, or a “pocket”) (see FIG. 4H) or may comprise a cylindrical (or other shaped) recess. Exemplary pins and plates can includes those described in U.S. Pat. No. 11,090,654, entitled “Multi-well separation apparatus and reagent delivery device,” incorporated herein by reference in its entirety for all purposes.

FIG. 4G shows an exemplary closed tip comprising a cylindrical recess at a distal end of the tip and a rim at a distal end of the tip. In some embodiments, the depth of the cylindrical recess is about 0.005 mm to about 0.1 mm, about 0.01 mm to about 0.1 mm, about 0.01 mm to about 0.2 mm, about 0.01 mm to about 0.3 mm, about 0.01 mm to about 0.4 mm, about 0.01 mm to about 0.5 mm, about 0.01 mm to about 0.6 mm, about 0.01 mm to about 0.7 mm, about 0.01 mm to about 0.8 mm, about 0.01 mm to about 0.9 mm, about 0.01 mm to about 1 mm, about 0.01 mm to about 2 mm, about 0.01 mm to about 3 mm, about 0.01 mm to about 4 mm, about 0.01 mm to about 6 mm, about 0.01 mm to about 7 mm, about 0.01 mm to about 8 mm, about 0.01 mm to about 9 mm, or about 0.01 mm to about 10 mm. In some embodiments, the tip comprises a rim at least partially enclosing a surface such as a flat surface. In some embodiments, the tip is flat with a rim and the rim height is between about 0.005 mm and about 7.5 mm. In some embodiments, the rim height is between about 0.01 mm and about 5 mm. In some embodiments, the rim height is about 0.02 mm, about 0.05 mm, about 0.1 mm, about 0.2 mm, about 0.5 mm, about 1 mm, about 2 mm, about 5 mm, or in a range between any of the aforementioned values. In some embodiments, the width of the rim is less than about 5 μm, about 5 μm to about 0.1 mm, about 0.01 mm to about 0.1 mm, about 0.01 mm to about 0.2 mm, about 0.01 mm to about 0.4 mm, about 0.01 mm to about 0.6 mm, about 0.01 mm to about 0.8 mm, about 0.01 mm to about 1 mm, about 0.01 mm to about 1.2 mm, about 0.01 mm to about 1.4 mm, about 0.01 mm to about 1.6 mm, about 0.01 mm to about 1.8 mm, about 0.01 mm to about 2.0 mm, about 0.01 mm to about 2.2 mm, about 0.01 mm to about 2.4 mm, about 0.01 mm to about 2.6 mm, about 0.01 mm to about 2.8 mm, about 0.01 mm to about 3.0 mm, about 0.01 mm to about 3.2 mm, about 0.01 mm to about 3.4 mm, about 0.01 mm to about 3.6 mm, about 0.01 mm to about 3.8 mm, about 0.01 mm to about 4 mm, about 0.01 mm to about 5 mm, or more than 5 mm. In some embodiments, the width of the rim is about 0.01 mm to about 3.8 mm.

In some embodiments, the tip comprises a flat surface with a rim.

In yet other variations, the closed tips may comprise one or more linear depressions. For example, the closed tips may comprise one linear depression (see FIGS. 4B and 4D), they may comprise two perpendicular linear depressions (see FIG. 4C), or they may comprise a plurality of linear depressions arranged in a grid-like arrangement. In other variations, the closed tips may comprise two parallel plates, between which the reagent may be held, or one or more capillaries within which the reagent may be held.

In other variations, the closed tips may comprise a concaved surface. The concaved surface may form a pocket, e.g., configured to at least partially hold one or more reagents. Such tips, such as a concaved tip, or a tip comprising a depression, may allow the reagent to be held more securely by the closed tip, for example by providing more surface area for adhesive forces between the closed tip and the reagent. FIG. 4H shows an exemplary closed tip comprising a distal end that is concaved. In some embodiments, the tip comprises a rim at least partially enclosing a concaved surface, forming a pocket and the depth of the pocket (e.g., from the rim to the bottom center of the pocket in the axial direction of the tip) can between about 0.001 mm and about 5 mm. In some embodiments, the pocket at the concaved distal end has a maximum depth. In some embodiments, the maximum depth of the concaved distal end is less than about 1 μm, about 1 μm to about 0.05 mm, about 1 μm to about 0.1 mm, about 1 μm to about 0.2 mm, about 1 μm to about 0.4 mm, about 1 μm to about 0.6 mm, about 1 μm to about 0.8 mm, about 1 μm to about 1 mm, about 1 μm to about 1.2 mm, about 1 μm to about 1.4 mm, about 1 μm to about 1.6 mm, about 1 μm to about 1.8 mm, about 1 μm to about 2.0 mm, about 1 μm to about 2.2 mm, about 1 μm to about 2.4 mm, about 1 μm to about 2.6 mm, about 1 μm to about 2.8 mm, about 1 μm to about 3.0 mm, about 1 μm to about 4 mm, about 1 μm to about 5 mm, about 1 μm to about 6 mm, about 1 μm to about 7 mm, about 1 μm to about 8 mm, about 1 μm to about 9 mm, about 1 μm to about 10 mm, or more than 10 mm. In some embodiments, the maximum depth of the concaved distal end is about 1 μm to about 5 mm.

The closed tip may have any suitable dimensions. In some embodiments, the cross-section of the close tips has a circular shape. In some variations, the largest cross-sectional diameter of the closed tips having a circular shape may be about 1 μm to about 10 μm, about 10 μm to about 100 μm, about 100 μm to about 1 mm, about 1 mm to about 10 mm, about 1 μm to about 1 mm, about 1 μm to about 10 mm, or larger than about 10 mm. In some embodiments, the largest cross-sectional diameter of the closed tips having a circular shape is about 100 μm to about 500 μm, about 100 μm to about 1 mm, about 100 μm to about 2 mm, about 100 μm to about 3 mm, about 100 μm to about 4 mm, about 100 μm to about 5 mm, about 100 μm to about 6 mm, about 100 μm to about 7 mm, about 100 μm to about 8 mm, about 100 μm to about 9 mm, about 100 μm to about 10 mm, about 500 μm to about 1 mm, about 500 μm to about 1.5 mm, about 500 μm to about 2 mm, about 500 μm to about 3 mm, about 500 μm to about 4 mm, about 500 μm to about 5 mm, about 500 μm to about 6 mm, about 500 μm to about 7 mm, about 500 μm to about 8 mm, about 500 μm to about 9 mm, about 500 μm to about 10 mm, about 1 mm to about 1.5 mm, about 1.5 mm to about 2 mm, about 2 mm to about 2.5 mm, or about 2.5 mm to about 3 mm. In some variations, the largest cross-sectional length of the closed tips having a square shape may be about 1 μm to about 10 μm, about 10 μm to about 100 μm, about 100 μm to about 1 mm, about 1 mm to about 10 mm, larger than about 10 mm, about 1 μm to about 10 mm, about 1 μm to about 1 mm, or about 1 mm to about 10 mm. In some embodiments, the total surface area for the cross-section of all pins on the plate may be less than about 1 μm², about 1 μm² to about 10 μm², about 1 μm² to about 10 μm², about 1 μm² to about 50 μm², about 1 μm² to about 100 μm², about 1 μm² to about 1 mm², about 10 μm² to about 100 μm², about 100 μm² to about 1 mm², about 1 mm² to about 10 mm², about 10 mm² to about 100 mm², or larger than 100 mm². It should be appreciated that each closed tip need not have the same configuration.

The closed tips may be closely or sparsely distributed across the plate. In some embodiments, there may be about 1 closed tip, about 2 closed tips, about 4 closed tips, about 8 closed tips, about 12 closed tips, about 16 closed tips, about 20 closed tips, or more than 20 closed tips per 10 cm² of plate area. In some embodiments, the total surface area for the cross-section of all pins on the plate is less than 1%, about 1%, about 2%, about 3%, about 4%, about 5%, about 6%, about 7%, about 8%, about 9%, about 10%, about 15%, about 20%, or about 25% relative to the area of the plate. The closed tips may be distributed with any spacing between a tip and its closest neighbor, such as less than 1 mm, about 1 mm, about 2 mm, about 3 mm, about 4 mm, about 5 mm, about 10 mm, about 15 mm, about 20 mm, about 25 mm, about 30 mm, about 40 mm, about 50 mm, about 60 mm, about 70 mm, about 80 mm, about 90 mm, about 100 mm, or more than 100 mm, or the spacing can be in a range between any of the aforementioned values.

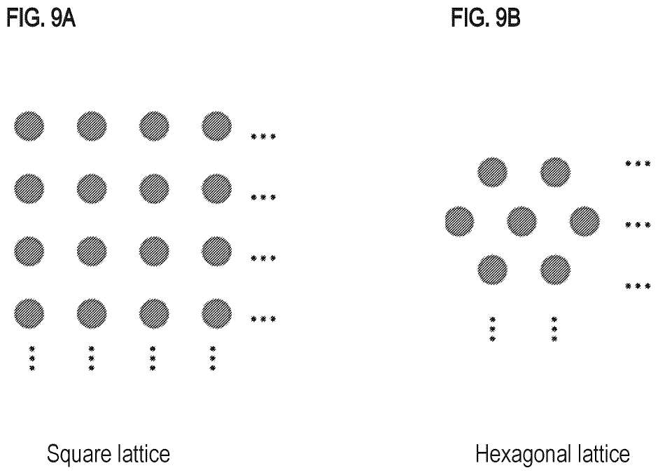

In some embodiments, the closed tips form a grid or lattice (e.g., as shown in FIG. 9A and FIG. 9B) on the distal surface (e.g., 1102) of the plate (e.g., 110) and the grid or lattice has a pitch (e.g., between the centers of two adjacent cross-section areas in a row or in a column) of between about 1 mm and about 100 mm, such as about 5 mm, about 10 mm, about 15 mm, about 20 mm, about 25 mm, about 30 mm, about 40 mm, about 50 mm, about 60 mm, about 70 mm, about 80 mm, about 90 mm, or about 100 mm. The grid or lattice can be symmetrical or asymmetrical.

The close tips may be evenly or unevenly distributed across the plate. In some embodiments, the closed tips are distributed evenly across the plate. In some embodiments, the evenly distributed closed tips form a two-dimensional array. The two-dimensional array may be selected from any of the two-dimensional Bravais lattice, such as a square lattice or a hexagonal lattice, as shown in FIGS. 9A-9B. In some embodiments, the closed tips are distributed unevenly across the plate. In some embodiments, the closed tips are distributed asymmetrically. In some embodiments, the asymmetrically distributed closed tips indicates the orientation of the pin array.

The surface of each closed tip may be smooth, or the surface may be rough (i.e., having surface irregularities). In some variations, the closed tips (e.g. closed tips 1202) may comprise the same plastic material as the stem, or one or more different materials. In some variations, the closed tips (e.g. closed tips 1202) may comprise a porous material, such as a polymer gel or a hydrogel (see FIGS. 4E-4F); an absorbent filling, such as polymer-based sponge, mesh, or in some variations may comprise a matrix, such as a dried fibrous structure, containing the reagent. For example, the closed tips may comprise cellulose (e.g., nitrocellulose, paper-like material), glass fibrous mesh, silk fibrous mesh, and the like. In some of these variations, all or a portion of the closed tip may be dissolvable when the closed tips of the pin array device (e.g. pin array device 100 or 200) are in contact with the target cell culture. In variations in which all or a portion of the closed tip 1202 is dissolvable, the dissolvable material may comprise any suitable material, such as but not limited to a salt, micro-particles, nano-particles, polyglycolide, poly(lactic acid), or poly(lactic-co-glycolic) acid co-polymer, or combinations thereof. In other variations, all or a portion of the closed tip 1202 may be meltable when the closed tips 1202 of the pin array device 100 are in contact with the target cell culture. In variations in which all or a portion of the closed tip 1006 is meltable, the dissolvable material may comprise any suitable material, such as but not limited to DMSO.

The closed tips (e.g., closed tips 1202) may each be located at the distal end of a stem (e.g., stem 1201 of pin array device 100 or 200). In some variations the closed tips may be integral to the stems, while in other variations, the closed tips may be attached to the stems in any suitable manner (e.g., using adhesives (glues, adhesive polymers, and the like), welding, mechanical fasteners, chemical bonding, a combination of these methods, or the like). The proximal ends of the stems may be connected to the plate, which may form an array of the first pins. For example, as shown in FIG. 2, the proximal ends of the stems 1202 may be connected to the distal surface 1102 of the plate 110 of pin array device 100, which may form an array of the first pins 120. In some variations the stems 1201 may be integral to the plate 110, while in other variations, the stems 1201 may be attached to the plate 110 in any suitable manner (e.g., using adhesives (glues, adhesive polymers, and the like), welding, mechanical fasteners, chemical bonding, a combination of these methods, or the like).

The stems (e.g., stem 1202 of reagent loading device 100 or 200) may have a length such that when the pin array device is in contact with the target cell culture, the closed tips (e.g., closed tips 1202) may be contacting the target cells at the surface of the target cell culture. For example, in some variations, the length of the stems may be about 1 mm to about 2 mm, about 2 mm to about 4 mm, about 4 mm to about 6 mm, about 6 mm to about 8 mm, about 8 mm to about 1 cm, about 1 cm to about 2 cm, about 2 cm to about 4 cm, about 4 cm to about 6 cm, longer than about 6 cm, about 1 mm to about 6 cm, about 1 mm to about 1 cm, or about 1 cm to about 6 cm. In some embodiments, the length of the stem allows stamping of the closed tips on the surface of a target cell culture.

The stems may have any suitable cross-sectional dimensions, which may be smaller or larger than the cross-sectional dimensions of the closed tips. In some variations, the largest cross-sectional dimension of the stems may be about 1 μm to about 10 μm, about 10 μm to about 100 μm, about 100 μm to about 1 mm, about 1 mm to about 10 mm, larger than about 10 mm, about 1 μm to about 10 mm, about 1 μm to about 1 mm, or about 1 mm to about 10 mm. It should be appreciated that in some variations the stems may have variable cross-sectional dimensions along their length (e.g., they may taper distally, taper proximally, or taper towards a midpoint). It should also be appreciated that each stem need not have the same configuration.

In some embodiments, the range of tip length is between about 2 mm and about 30 mm, the range of tip diameter is between about 0.5 mm and about 8 mm, the rim height can be between about 0.01 mm and about 5 mm with a rim width of between about 0.01 mm and about 3.8 mm (e.g., when the tip is flat with rim), the depth of the pocket (rim to bottom of the pocket) can be between 0.001 mm and about 5 mm (e.g., when the tip has a concaved surface), and the spacing of tips is between about 1 mm and about 100 mm.

In some embodiments, the density of first pins per unit area on the plate can be between about 0.0001 and about 100 pins per mm². In some embodiments, the density of first pins per unit area on the plate can be about 0.001 pins per mm², about 0.005 pins per mm², 0.01 pins per mm², 0.05 pins per mm², 0.1 pins per mm², 0.5 pins per mm², 1 pin per mm², 2 pins per mm², 5 pins per mm², 10 pins per mm², 20 pins per mm², 50 pins per mm², 100 pins per mm², or in a range between any of the aforementioned values. Compared to paper disks which are bulky and not suitable for high throughput assays (generally only about 5 paper disks in an entire dish), the present pin array device allows more efficient, accurate, and high throughput analysis.

In some embodiments, the amount of reagent on the closed tip of each pin can be precisely controlled, providing an advantage over antibiotic-containing paper disks when it is challenging to control the amount of reagents in the paper disks. In some embodiments, the reagent can be dried on the tip, e.g., via lyophilization or other drying methods, and there is no need to reconstitute the reagent in water or a solution. Thus, unlike the antibiotic-containing paper disks which must be wetted and manually placed into a dish, the present pin array devices and methods facilitates automation and reduction of the amount of reagents needed. Further, due to the ability to use a relatively uniform array of pins and control reagent amount on each tip, assay variations can be reduced.

c. Second Pin (Orientation Pin)

The pin array devices may optionally comprise orientation features that may promote the pin array device contacting target cell cultures in a particular orientation. In some variations, the orientation features may be indicators of orientation, thus providing the user information that allows the user to readily recognize the configuration of experiment.

In some variations, the orientation features may be a second pin (orientation pin) that marks the orientation of the pin array. In some variations, the second pin may be integral to the pin array devices (e.g., to the plate of the pin array devices described herein). In some variations, one or more orientation pins can be used to easily tell what drug is at which pin location no matter how the image is taken, and whether the image is flipped or not during image processing. The one or more orientation pins can also function as first pins that hold one or more reagents (e.g., antibiotics), for instance, the pattern of first pins on the plate can be asymmetrical, with one or more first pins having no corresponding “mirror image” first pin(s) along an axis or when the plate is rotated by a certain degree (e.g., around the central point of the plate).

FIGS. 2 and 3 show one variation of a reagent loading device having an orientation feature comprising a second pin 130 as an indicator of orientation. As shown there, the second pin 130 may be attached to the proximal surface 1102 of the plate 110.

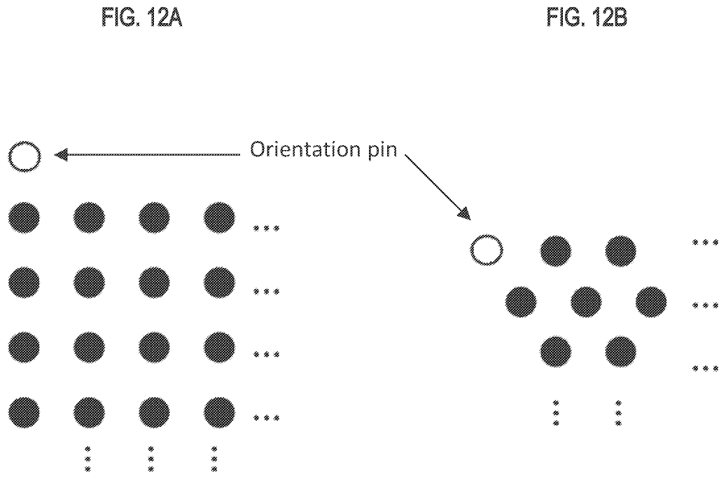

It should be appreciated that the second pin (orientation pin) of the pin array device may have any number of configurations or other physical shapes, such as half circles, angled slots, bent or curved slots, triangles, crescents, parallelograms, or the like. In some variations, the second pin may be located outside of the pin array comprising the first pins. FIGS. 12A-12B show additional examples of the position of orientation pin in relation to the first pins.

d. Containment Element

In some variations, the pin array device may further comprise a containment element. The containment element may be configured to protect the compositions comprising an agent loaded on the tips of the first pins of the pin array device, while also being configured such that that it can be removed from the pin array device while leaving the compositions on the tips of the first pins. In some variations, the containment element may comprise a substantially planar surface. In some variations, when the containment element is placed in a position in which it protects the compositions on the tips of the first pins, it is not in contact with the compositions.

In some variations, the pin array device may have a design configured to protect the compositions loaded on the tips of the first pins of the pin array devices with a separate containment element, such as a protective dish. The protective dish may form the bottom parts of the pin array device. One such variation is shown in FIG. 3. As shown there, the pin array device 200 may comprise a protective dish 210, wherein the dish comprises a bottom plate and a second rim 2103 disposed on the bottom plate 2101, wherein the bottom plate of the dish comprises a proximal surface 2101 and a distal surface 2102, and optionally wherein the second rim is disposed on the proximal surface of the bottom plate 2101. In some variations, the plate 110 is capable of fittingly engage with the dish 210. In some variations, the plate 110 forms a tight fit with the protective dish 210. In some variations, the plate and the dish do not rotate relative to each other when a tight fit is formed.

In some variations, the protect dish (e.g. the protective dish 210 of the pin array device 200) is configured such that when a tight fit is formed between the plate (e.g. plate 110 of the pin array device 200) and the protect dish (e.g. the protective dish 210 of the pin array device 200), the lengths of the first pins are shorter than the distance between the distal surface of the plate and the proximal surface of the bottom plate of the protective dish when the plate and the protective dish fittingly engage each other, thereby preventing the tips of the first pins from touching the bottom plate. In some variations, the second pin is longer than the first pins, thereby preventing the tips of the first pins from touching the bottom plate when the plate and the protective dish fittingly engage each other. In some variations, the device further comprises a third pin on the proximal surface of the bottom plate of the protective dish that is longer than the first pins, thereby preventing the tips of the first pins from touching the bottom plate when the plate and the protective dish fittingly engage each other. In some variations, the length of the second pin and/or the length of the third pin is about the same as the distance between the distal surface of the plate and the proximal surface of the bottom plate when the plate and the protective dish fittingly engage each other, thereby preventing the tips of the first pins from touching the bottom plate.

### II. Composition/Agent

Each of the tips of the first pins of the pin array device described herein may be loaded with composition comprising an agent. The composition/agent may be in any suitable form, such as but not limited to a liquid (including an emulsion), a solution, a gel, or a solid (such as a powder or a film). When the composition/agent is in a liquid or solution form, the composition/agent may adhere to each tip due to cohesive forces within the liquid (i.e., surface tension) and adhesive forces between the liquid and the tip of the first pins. The volume of the liquid or solution that may adhere to each tip of the first pins may in some variations be about 1 pL to about 10 pL, about 10 pL to about 100 pL, about 100 pL to about 1 nL, about 1 nL to about 10 nL, about 10 nL to about 100 nL, about 100 nL to about 1 μL, about 1 μL to about 10 μL, or more than about 10 μL, depending on the configuration and material of the tips of the first pins and the material properties of the liquid or solution. When the composition/agent is in a solid form, the composition/agent may adhere to each tip due to cohesive forces within the solid and adhesive forces (i.e. static force) between the solid and the tip of the first pins. The weight of the solid that may adhere to each tip of the first pins may in some variations be about 0.1 ng to about 1 ng, about 1 ng to about 10 ng, about 10 ng to about 100 ng, about 100 ng to about 1 μg, about 1 μg to about 10 μg, about 10 μg to about 100 μg, about 100 μg to about 1 mg, about 1 mg to about 10 mg, about 10 mg to about 100 mg, or more than 100 mg, depending on the configuration and material of the tips of the first pins and the material properties of the solid.

While in some variations the tips of the first pins described herein may be loaded with the same composition comprising an agent, it may often be desirable to load the tips of the first pins with different compositions/agents. For example, it may be desirable to do so in order that the target cell culture may be subject to different compositions/agents. In another variation, it may be desirable such that some of the first pins are not loaded with a composition/agent while the others are loaded. The composition/agent may comprise, but are not limited to proteins, nucleic acids, cells, microorganisms (e.g., bacteria, fungi), plants (e.g., algae), viruses, small molecule drugs or any chemical compounds. In some variations, the tips of the first pins may be loaded with a particular library of reagents desired to be tested. For example, the pin array device may be loaded with a bacterial library, a drug library (e.g., a kinase inhibitor library), an antibody library, or the like. In some variations, the pin array device may be loaded with antibiotic drugs.

### III. Target Cell Culture

As mentioned above, the pin array devices described herein may be used on a target cell culture. The target cell culture may be in any suitable form. In some variations, the target cell culture is in a solid form, for example, on the surface of a solid culture media, a gel, a membrane, a container, or a slide. The target cell may be of any type of interest, including but are not limited to, bacteria, yeast, fungi, or mammalian cells. In some variations, the target cell culture is a microbiome culture. In some variations, the target cell culture is a bacteria culture. In some variations, the target cell culture is a bacteria lawn on the surface of a solid culture media (e.g. an LB agar media). In some embodiment, the target cells are collected from a tissue from a subject. The target cell culture may be prepared by any desired method common in the art, such as using plating methods. In some embodiments, the target cells are pre-cultured prior to application onto the culture media.

In some embodiments, the target cell culture have a pin array applied thereto. In some embodiments, the target cell culture reacts with the pin array comprising one or more compositions. In some embodiments, the target cell culture exhibits differences before and after the pin array device is applied, including but are not limited to, differences that are visually, microscopically, spectrally, or fluorescently detectible. In one specific embodiment wherein the first pins of the pin array device are loaded with one or more compositions comprising an antibiotic drug, wherein the target cell culture is a bacteria culture, and wherein the closed tips of the first pins are stamped onto the surface of the bacteria culture, the bacteria culture in the vicinity of the closed tips of the first pins may be inhibited or eliminated while the bacteria not in the vicinity of the closed tips remain viable, thereby producing a pattern on the bacteria culture with inhibition zones around the positions stamped by the closed tips.

### IV. Computer Systems

The present disclosure provides computer systems that are programmed to implement and/or be used in connection with devices and methods of the disclosure. In some aspects, provided herein a computer system that is programmed or otherwise configured to implement methods of screening compositions or agents, for instance, by interfacing with a potential user or a user of the pin array device, kit, or system disclosed herein. The computer system can regulate various aspects of the present disclosure, such as, for example, controlling the installation, operation, maintenance, data collection (such as image collection), data processing (such as image processing), and/or replacement of the pin array apparatus, device, kit, or system disclosed herein. The computer system can be an electronic device of a user or a computer system that is remotely located with respect to the electronic device. The electronic device can be a mobile electronic device.

In some embodiments, the computer system comprises a central processing unit (CPU), which can be a single core or multi core processor, or a plurality of processors for parallel processing. In some embodiments, the computer system comprises memory or memory location (e.g., random-access memory, read-only memory, flash memory), electronic storage unit (e.g., hard disk), communication interface for communicating with one or more other systems, and peripheral devices, such as cache, other memory, data storage and/or electronic display adapters. In some embodiments, the memory, storage unit, interface and peripheral devices are in communication with the CPU through a communication bus such as a motherboard. In some embodiments, the storage unit is a data storage unit or data repository for storing data. In some embodiments, the computer system is operatively coupled to a computer network with the aid of the communication interface. In some embodiments, the network is the Internet, an internet and/or extranet, or an intranet and/or extranet that is in communication with the Internet. In some embodiments, the network is a telecommunication and/or data network. In some embodiments, the network comprises one or more computer servers, which can enable distributed computing, such as cloud computing. The network, in some cases with the aid of the computer system, can implement a peer-to-peer network, which may enable devices coupled to the computer system to behave as a client or a server.

In some embodiments, the CPU of the computer system can execute a sequence of machine-readable instructions, which can be embodied in a program or software. The instructions may be stored in a memory location, such as the memory. The instructions can be directed to the CPU, which can subsequently program or otherwise configure the CPU to implement methods of the present disclosure. The CPU can be part of a circuit, such as an integrated circuit. One or more other components of the system can be included in the circuit.

In some embodiments, the storage unit can store files, such as drivers, libraries, and saved programs. The storage unit can store user data, e.g., user preferences and user programs. The computer system in some cases can include one or more additional data storage units that are external to the computer system, such as located on a remote server that is in communication with the computer system through an intranet or the Internet.

In some embodiments, the computer system communicates with one or more remote computer systems through the network, including a local area network (“LAN”); wide area network (“WAN”), such as the Internet; metropolitan area network (“MAN”); point-to-point or peer-to-peer connection; etc. Communication with other devices may be accomplished using any suitable networking protocol. For example, one suitable networking protocol may include the Internet Protocol (“IP”), Transmission Control Protocol (“TCP”), User Datagram Protocol (“UDP”), or combinations thereof, such as TCP/IP or UDP/IP.

In some embodiments, the computer system communicates with a remote computer system of a user (e.g., a cloud computing system, a server system). Examples of remote computer systems include personal computers (e.g., portable PC), slate or tablet PC's (e.g., Apple® iPad, Samsung® Galaxy Tab), telephones, Smart phones (e.g., Apple® iPhone, Android-enabled device, Blackberry®), or personal digital assistants. The user can access the computer system via the network.

In some embodiments, the computer system comprises a front end. In some embodiments, the front end is a native application (app) for a particular operating system or a web app. In some embodiments, the native app is a mobile app for e.g., Google, Android, or iOS. In some embodiments, the front end uses a JavaScript framework. In some embodiments, the front end platform links the app to a cloud service. In some embodiments, the front end is based on Angular. In some embodiments, the cloud service is based on Azure. In some embodiments, the system further comprises a back end. In some embodiments, the back end is a cloud service. In some embodiments, the cloud back end service comprises data storage, security and/or processing. In some embodiments, the app is a preconfigured app. In some embodiments, the preconfigured app comprises a user-friendly interface for a computer or mobile device, such as a companion web app.

Methods as described herein can be implemented by way of machine (e.g., computer processor) executable code stored on an electronic storage location of the computer system, such as, for example, on the memory or electronic storage unit. The machine executable or machine-readable code can be provided in the form of software. During use, the code can be executed by the processor. In some cases, the code can be retrieved from the storage unit and stored on the memory for ready access by the processor. In some situations, machine-executable instructions are stored on memory. The code can be pre-compiled and configured for use with a machine having a processer adapted to execute the code or can be compiled during runtime. The code can be supplied in a programming language that can be selected to enable the code to execute in a pre-compiled or as-compiled fashion.

Aspects of the systems and methods provided herein, such as the computer system, can be embodied in programming. Various aspects of the technology may be thought of as “products” or “articles of manufacture” typically in the form of machine (or processor) executable code and/or associated data that is carried on or embodied in a type of machine readable medium. Machine-executable code can be stored on an electronic storage unit, such as memory (e.g., read-only memory, random-access memory, flash memory) or a hard disk. “Storage” type media can include any or all of the tangible memory of the computers, processors or the like, or associated modules thereof, such as various semiconductor memories, tape drives, disk drives and the like, which may provide non-transitory storage at any time for the software programming. All or portions of the software may at times be communicated through the Internet or various other telecommunication networks. Such communications, for example, may enable loading of the software from one computer or processor into another, for example, from a management server or host computer into the computer platform of an application server. Thus, another type of media that may bear the software elements includes optical, electrical, and electromagnetic waves, such as used across physical interfaces between local devices, through wired and optical landline networks and over various air-links. The physical elements that carry such waves, such as wired or wireless links, optical links, or the like, also may be considered as media bearing the software. As used herein, unless restricted to non-transitory, tangible “storage” media, terms such as computer or machine “readable medium” refer to any medium that participates in providing instructions to a processor for execution.

Such processors may comprise, or may be in communication with, media, for example one or more non-transitory computer-readable media, that may store processor-executable instructions that, when executed by the processor, can cause the processor to perform methods according to this disclosure as carried out, or assisted, by a processor. Carrier-wave transmission media may take the form of electric or electromagnetic signals, or acoustic or light waves such as those generated during radio frequency (RF) and infrared (IR) data communications. Examples of non-transitory computer-readable medium may include, but are not limited to, an electronic, optical, magnetic, or other storage device capable of providing a processor, such as the processor in a web server, with processor-executable instructions. Other examples of non-transitory computer-readable media include, but are not limited to, a floppy disk, CD-ROM, magnetic disk, memory chip, ROM, RAM, ASIC, configured processor, all optical media, all magnetic tape or other magnetic media, or any other medium from which a computer processor can read. Many of these forms of computer readable media may be involved in carrying one or more sequences of one or more instructions to a processor for execution. The processor, and the processing, described may be in one or more structures, and may be dispersed through one or more structures. The processor may comprise code to carry out methods (or parts of methods) according to this disclosure.

Methods and systems of the present disclosure can be implemented by way of one or more algorithms. An algorithm can be implemented by way of software upon execution by the central processing unit. The algorithm can, for example, determine sizes of the zone of inhibition on the target cell culture upon contacting the pin array loaded with one or more agents.

In some embodiments, the computer system can be or is in communication with an image capturing device, such as a digital camera, to collect images of the target cell culture upon contacting the pin array loaded with one or more agents.

In some embodiments, the computer system can be or is in communication with an electronic display that comprises a user interface (UI) for providing, for example, an interface to start and/or monitor the progress of image collection of target cell culture at various time points and image processing of the target cell culture. Examples of UI's include, without limitation, a graphical user interface (GUI) and web-based user interface.

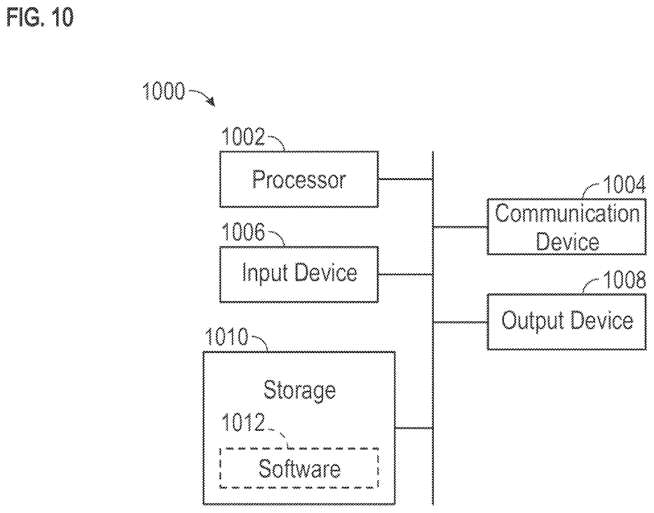

Referring to FIG. 10, device 1000 can be a host computer connected to a network. Device 1000 can be a client computer or a server. The device can be any suitable type of microprocessor-based device, such as a dedicated computing device, a personal computer, work station, server, handheld computing device (portable electronic device) such as a phone or tablet, an edge-AI device, or a neural network device. The device can include, for example, one or more of processors 1002, input device 1006, output device 1008, storage 1010, and communication device 1004. Input device 1006 and output device 1008 can generally correspond to those described above and can either be connectable or integrated with the computer. Input device 1006 can be any suitable device that provides input, such as a camera sensor, touchscreen, keyboard or keypad, mouse, or voice-recognition device. Output device 1008 can be any suitable device that provides output, such as an illuminator, a touchscreen, haptics device, or speaker. Storage 1010 can be any suitable device that provides storage, such as an electrical, magnetic, or optical memory including a RAM, cache, hard drive, or removable storage disk. Communication device 1004 can include any suitable device capable of transmitting and receiving signals over a network, such as a network interface chip or device. The components of the computer can be connected in any suitable manner, such as via a physical bus, or wirelessly.

Software 1012, which can be stored in storage 1010 and executed by processor 1002, can include, for example, the programming that embodies the functionality of the present disclosure (e.g., as embodied in the devices described above). Software 1012 can also be stored and/or transported within any non-transitory, computer-readable storage medium for use by or in connection with an instruction execution system, apparatus, or device, such as those described above, that can fetch instructions associated with the software from the instruction execution system, apparatus, or device and execute the instructions. In the context of this disclosure, a computer-readable storage medium can be any medium, such as storage 1010, that can contain or store programming for use by or in connection with an instruction-execution system, apparatus, or device. Software 1012 can also be propagated within any transport medium for use by or in connection with an instruction-execution system, apparatus, or device, such as those described above, that can fetch instructions associated with the software from the instruction-execution system, apparatus, or device and execute the instructions. In the context of this disclosure, a transport medium can be any medium that can communicate, propagate, or transport programming for use by or in connection with an instruction-execution system, apparatus, or device. The transport readable medium can include, but is not limited to, an electronic, magnetic, optical, electromagnetic, or infrared wired or wireless propagation medium.

Device 1000 may be connected to a network, which can be any suitable type of interconnected communication system. The network can implement any suitable communications protocol and can be secured by any suitable security protocol. The network can comprise network links of any suitable arrangement that can implement the transmission and reception of network signals, such as wireless network connections, T1 or T3 lines, cable networks, DSL, or telephone lines. Device 1000 can implement any operating system suitable for operating on the network. Software 1012 can be written in any suitable programming language. In various embodiments, application software embodying the functionality of the present disclosure can be deployed in different configurations, such as in a client/server arrangement or through a Web browser as a Web-based application or Web service, for example.

### V. Kits

It should be appreciated that the components of the pin array devices described herein may, in addition to having the form of devices, have the form of kits for biological or chemical assays. Described herein are additionally kits and/or systems for assays comprising any of the pin array devices (e.g., the pin array device 100 described above). The pin array device may comprise a plurality of first pins, and a plate having a proximal surface and a distal surface, wherein the plurality of first pins are integral to the plate and are on the distal surface of the plate. In some variations, the plurality of first pins form an array, such that each of the plurality of pins of the pin array device may be configured to simultaneously contact a target cell culture. In some embodiments, the plurality of first pins are pre-loaded with one or more compositions comprising an agent. In some variations, the device further comprises a second pin. In some variations, the second pin is capable of marking the orientation of the array, such that the exact configuration of the composition and/or agents loaded onto the tips of the first pins may be readily known from the second pin (orientation pin). In some variations, the second pin is outside of the array.

In some variations, the kits and/or systems may comprise the pin array device described herein, wherein the plate of the pin array device is round, and wherein the plate further comprises a first rim on the distal surface of the plate.

The kit and/or system may optionally comprise a protective dish, wherein the dish comprises a bottom plate and a second rim disposed on the bottom plate, wherein the bottom plate of the dish comprises a proximal surface and a distal surface. In some variations, the plate is capable of fittingly engage the protective dish, and wherein the plate and the dish does not rotate relative to each other when a tight fit is formed.

In some variations of the kits disclosed herein, the length of the first pins is shorter than the distance between the distal surface of the plate and the proximal surface of the bottom plate when the plate and the protective dish fittingly engage each other, thereby preventing the tips of the first pins from touching the bottom plate. In some variations, the second pin is longer than the first pins, or the device further comprises a third pin on the proximal surface of the plate that is longer than the first pins. In some variations, the length of the second pin and/or the length of the third pin is about the same as the distance between the distal surface of the plate and the proximal surface of the bottom plate when the plate and the protective dish fittingly engage each other, thereby preventing the tips of the first pins from touching the bottom plate.

In some variations, the kit may further comprise one or more containers in which the target cell cultures and/or culture media may be prepared. Any suitable containers may be used, such as petri dishes. In some variations, the containers may be configured to fittingly engage the pin array device, wherein the container and the plate of the pin array device do not rotate relative to each other when a tight fit is formed. In some variations, the kit further comprise one or more culture media pre-packaged in the containers. In some variations, the kit further comprise one or more target cell cultures pre-packaged in the containers.

The system may further optionally comprise one or more libraries of compositions comprising an agent. In some embodiments, the compositions may be loaded manually on to individual closed tips in any pattern desired. In some embodiments, the compositions are pre-packaged in microwell plates that matches the configuration of the closed tips of the first pins, and the closed tips may be loaded with the compositions simultaneously (e.g. by dipping the closed tips into wells of the microwell plates).

### VI. Methods

Also described herein are methods of using the pin array devices or kits, or systems, described here. Generally, a target cell culture (e.g. a bacteria) may be prepared (e.g. in a dish). Once the target cell culture is prepared, the plurality of the first pins (e.g. first pins 120 described above) of pin array devices (e.g. pin array device 100 described above) comprising one or more compositions at the tips (e.g. tips 1202 described above) comprising an agent may be coupled to the target cell culture, wherein the tips of the first pins are in contact with the target cell culture, and wherein the effects of the composition/agent on the target cell culture are analyzed.

In some variations, the pin array device described herein may be pre-loaded with a reagent or test agent on each of the plurality of tips of the first pins. In other variations, the user may load each of the plurality of closed tips with a composition/agent. When composition/agent is in liquid or solution form, this may be done by dipping each of the closed tips into the liquid or solution, and then removing them from the liquid or solution. In some variations, the pin array device may be dipped into isolated areas comprising the compositions/agents in a configuration corresponding to the configuration of the first pins. This may allow each tip of the first pins to be simultaneously loaded with reagent or test agent, even when the compositions/agents to be loaded on one or more of the tips of the first pins are different. In other variations, one or more of the tips of the first pins may be individually dipped into a liquid or solution to load the compositions/agents. In some variations, a defined volume of liquid or solution may be applied to each tip of the first pins. In some of these variations, the tip design may comprise a depression or other surface feature (e.g., a hemispherical depression, cylindrical recess, or one or more linear depressions, or a space between two parallel plates, or one or more capillaries, as described above with respect to FIGS. 4A-4H), which may be configured to hold a particular volume of a given reagent. For example, the depression or other surface design of the closed tips may have dimensions configured such that when the tip is dipped into the composition/agent, a defined volume is deposited or trapped at the closed tip, depending on the surface tension and surface affinity of the reagents (e.g., media, phosphate buffered saline, DMSO).

In instances when the composition is in a solid (e.g., powder) form, the tips of the first pins described herein may be loaded with the reagent in solution in the same manner as a reagent in a liquid or solution form, and the liquid in the solution may then be allowed to evaporate, leaving a solid reagent remaining on the tips of the first pins. When the reagent is a cell or microorganism, in some variations the tips of the first pins may be loaded with the cells or microorganisms by being loaded with droplets of suspensions containing the cells or microorganism in cryostorage solution in the same manner as a reagent in liquid or solution form, and the pin array device may then be frozen. It should be appreciated that not all tips of the first pins need be loaded with a reagent (e.g., some tips of the first pins may not be loaded with a reagent so as to provide a control condition).

In some instances, the closed tips described herein may be loaded with a gel, such as but not limited to a hydrogel or a sol-gel. In some cases, the tips may be directly loaded with a gel. In other cases, the tips may be loaded with a liquid, which may then be cured to form a gel (e.g., polymerization may be light-induced, chemically induced, thermally induced, or the like). In yet other cases, the closed tips may be loaded with a liquid, which may then at least partially evaporate to leave behind a gel. In some of these variations, the reagent or test agent may be in a gel form, while in other variations the reagent or test agent may be incorporated into a gel (i.e., the gel may immobilize the reagent or test agent). In these variations, non-limiting examples of the reagent or test agent may comprise proteins, nucleic acids, cells, microorganisms (e.g., bacteria, fungi), plants (e.g., algae), viruses, small molecule drugs or any chemical compounds, or a particular library of reagents desired to be tested (e.g., a bacterial library, a drug library (e.g., a kinase inhibitor library), an antibody library, a virus library, a gene library, a polymer library, a peptide library, a cell library, or the like).

The loaded pin array devices described herein (e.g. pin array devices 100) may be lowered into the target cell culture such that the first pins of the pin array devices (e.g. first pins 120) are in contact with the target cells. In variations in which the system comprises a second pin (orientation pin), the second pin may be used to readily know the exact configuration of the composition and/or agents loaded onto the tips of the first pins. In some embodiments, the target cell culture exhibits differences before and after the pin array device is applied, including but are not limited to differences that are visually, microscopically, spectrally, or fluorescently detectible. In some variations, the target cell culture has visual differences before and after the pin array device is applied, including but are not limited to, changes in color, density, texture, or distribution. In some embodiments, the target cell culture is inhibited upon contacting one or more compositions loaded onto the first pins. In some embodiments, inhibition zones may be observed (similar to Kirby-Bauer disc diffusion test).

In some embodiments, the effects of the agents are analyzed while the plurality of first pins are in contact with the target cell culture. In some embodiments, the effects of the agents are analyzed after the plurality of the first pins are separated from the target cell culture.

After the compositions are delivered to the target cell culture, a sufficient period of time may be allowed to elapse such that any desired reactions may occur. In some embodiments, the target cell culture may exhibit instant changes detectible by a desired method. In some embodiments, the pin array device may be in contact with the target cell culture for about 1 minute, about 5 minutes, about 10 minutes, about 15 minutes, about 30 minutes, about 1 hour, about 2 hours, about 4 hours, about 8 hours, about 12 hours, about 16 hours, about 20 hours, about 24 hours, about 36 hours, about 48 hours, about 72 hours, about 96 hours, about 120 hours, or more than about 120 hours before analysis. In some embodiments, the target cell culture may be incubated after the pin array device is removed for about 15 minutes, about 30 minutes, about 1 hour, about 2 hours, about 4 hours, about 8 hours, about 12 hours, about 16 hours, about 20 hours, about 24 hours, about 36 hours, about 48 hours, about 72 hours, about 96 hours, about 120 hours, or more than about 120 hours before analysis. After the assaying processes are carried out, the results may be analyzed using any known techniques, such as by identifying sizes of inhibition zone (similar to Kirby-Bauer disc diffusion test), detecting color changes in the target cell culture, detecting microscopic changes in the target cell culture, or detecting fluorescent changes in the target cell culture. In some embodiments, the plurality of the first pins are in contact with the target cell culture for at least about 30 minutes before the effects of the agents on the target cell culture are analyzed. The analysis may be carried out using any suitable methods and devices, such as the computer systems in Section IV.

In a non-limiting example, a pin array device may be used to deliver one or more antimicrobial agents to a gel (e.g., an agar gel) having a bacteria cultured on its surface or that will later have a bacteria cultured on its surface. In one specific variation wherein the first pins of the pin array device are loaded with one or more compositions comprising an antibiotic drug, wherein the target cell culture is a bacteria culture, and wherein the closed tips of the first pins are stamped onto the surface of the bacteria culture, the bacteria culture in the vicinity of the closed tips of the first pins is inhibited or eliminated while the bacteria not in the vicinity of the closed tips remain viable, thereby producing a pattern on the bacteria culture with inhibition zones around the positions stamped by the closed tips. The antibacterial effects of agents may be analyzed by comparing the sizes of inhibition zones, wherein larger area of inhibition zones indicate stronger antibacterial effects.

The pin array device may be disposable, such that it is configured for a single use. Thus, after completion of the desired processes, it may be discarded. In other variations, the pin array device may be reused by re-loading the plurality of first pins with one or more compositions comprising an agent.

### EXAMPLES

Specific details are given in the above description to provide a thorough understanding of the embodiments. However, it is understood that the embodiments may be practiced without these specific details. Those skilled in the art can readily recognize that numerous variations and substitutions may be made in the disclosed embodiments, their use and their configuration to achieve substantially the same results as achieved by the embodiments described herein. Accordingly, there is no intention to limit the claims to the disclosed exemplary forms. Many variations, modifications and alternative constructions fall within the scope and spirit of the disclosure as expressed in the claims.

### Example #1

As one example, the methods described herein was used to study the antibiotic susceptibility of bacteria using the pin array device.

1. Cell Introduction: E. coli resuspension were plated onto LB agar plates and incubated at 37° C. until the cells reach desired condition.

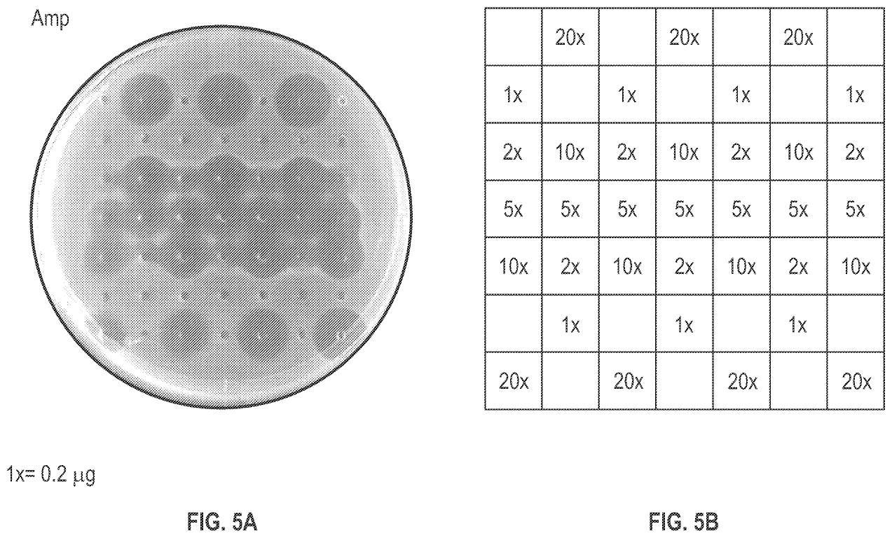

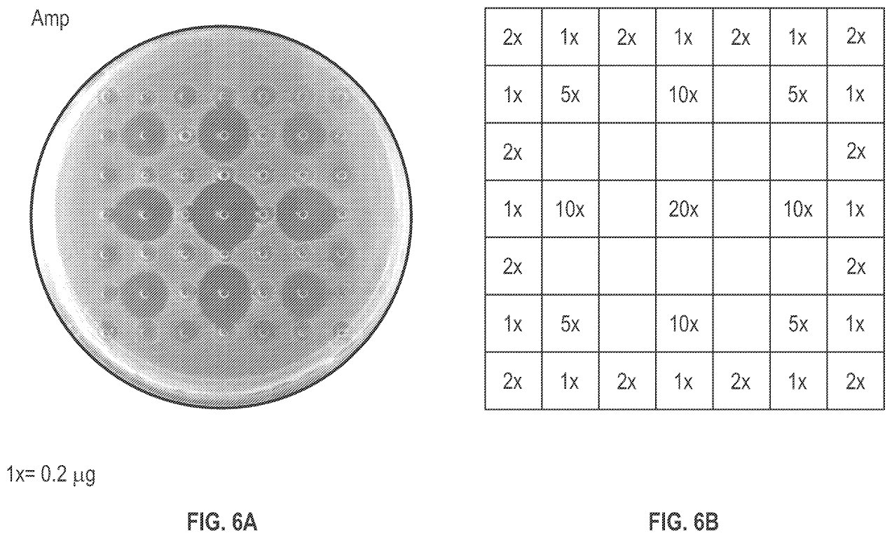

2. Pin array preparation: Two 7×7 pin arrays were loaded with ampicillin from 0.2 μg to 4 μg according to the configurations shown in FIGS. 5B and 6B, respectively.

3. Treatment: After the cells reach the desired condition, the pin arrays loaded with ampicillin according to the configurations shown in FIGS. 5B and 6B were coupled to LB agar plates with E. coli culture. The LB agar plates were incubated at 37° C.

4. Results and analysis: The results are shown in FIGS. 5A and 6A, respectively. As shown in FIG. 5A, zones of inhibition by ampicillin are well-defined, and a dose-dependent manner of the sizes of the zones can be clearly observe. Similarly, in FIG. 6A, well-defined, dose-dependent zones of inhibition can be observed.

### Example #2

As another example, the methods described herein was used to study the antibiotic susceptibility of bacteria and short-term storage stability of the assay.

1. Cell Introduction: E. coli resuspension were plated onto LB agar plates and incubated at 37° C. until the cells reach desired condition.

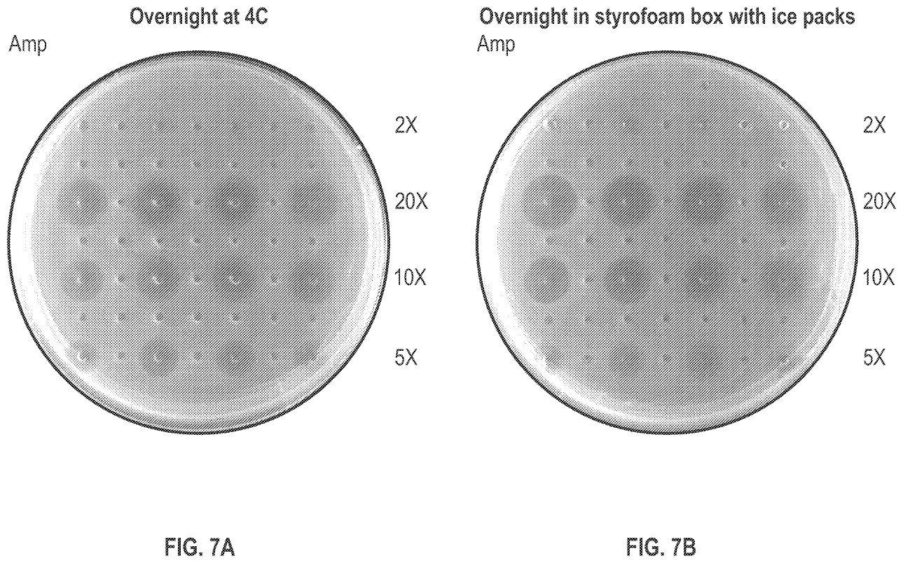

2. Pin array preparation: Two 7×7 pin arrays were loaded with ampicillin from 0.4 μg to 4 μg according to the configurations shown in FIGS. 7A and 7B, respectively.

3. Treatment: After the cells reach the desired condition, the pin arrays loaded with ampicillin according to the configurations shown in FIGS. 7A and 7B were coupled to LB agar plates with E. coli culture. The LB agar plates were incubated at 37° C., and stored overnight at 4° C. and in Styrofoam box with ice packs, respectively.

4. Results and analysis: The results are shown in FIGS. 7A and 7B, respectively. Well-defined, dose-dependent zones of inhibition can be observed in both FIGS. 7A and 7B, demonstrating feasibility of the pin array device describe herein in studying the antibiotic susceptibility of bacteria and stability for short-term storage.

### Example #3

As another example, the methods described herein was used to study the antibiotic susceptibility of bacteria and long-term storage stability of the assay.

1. Cell Introduction: E. coli resuspension were plated onto LB agar plates and incubated at 37° C. until the cells reach desired condition.

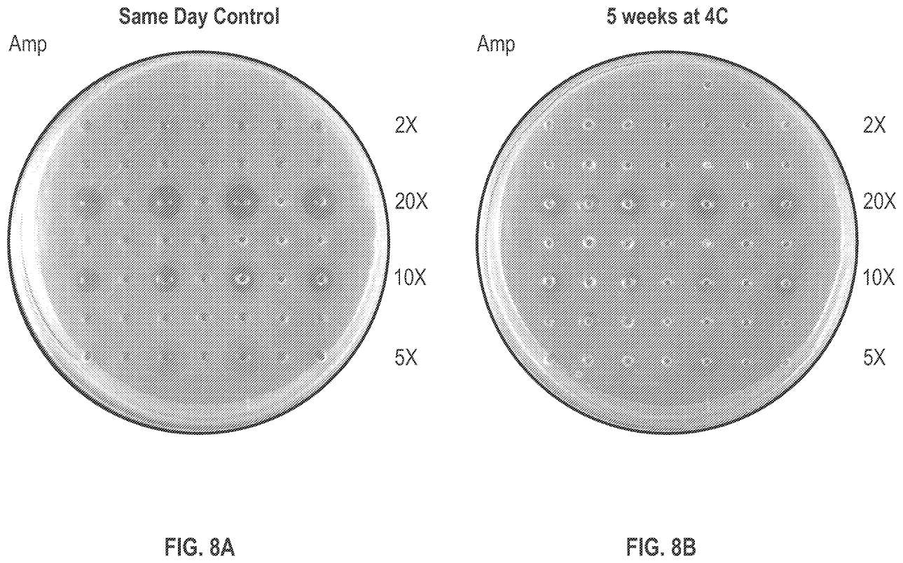

2. Pin array preparation: A 7×7 pin array was loaded with ampicillin from 0.4 μg to 4 μg according to the configurations shown in FIG. 8A.

3. Treatment: After the cells reach the desired condition, the pin array loaded with ampicillin according to the configurations shown in FIG. 8A was coupled to an LB agar plate with E. coli culture. The LB agar plate was incubated at 37° C., and images of the plates were taken at the same day (FIG. 8A), and after 5 weeks at 4° C. (FIG. 8B).

4. Results and analysis: The results are shown in FIGS. 8A and 8B, respectively. Well-defined, dose-dependent zones of inhibition can be observed in FIG. 8A. Albeit smaller zones of inhibition were observed in FIG. 8B after 5 weeks of storage at 4° C., zones of inhibition were still discernable in a dose-dependent manner. These results demonstrate feasibility of the pin array device describe herein in studying the antibiotic susceptibility of bacteria and stability for long-term storage.

### Example #4

As another example, the methods described herein was used to study the antibacterial effects of combinations of compounds.

1. Cell Introduction: E. coli resuspension were plated onto LB agar plates and incubated at 37° C. until the cells reach desired condition.

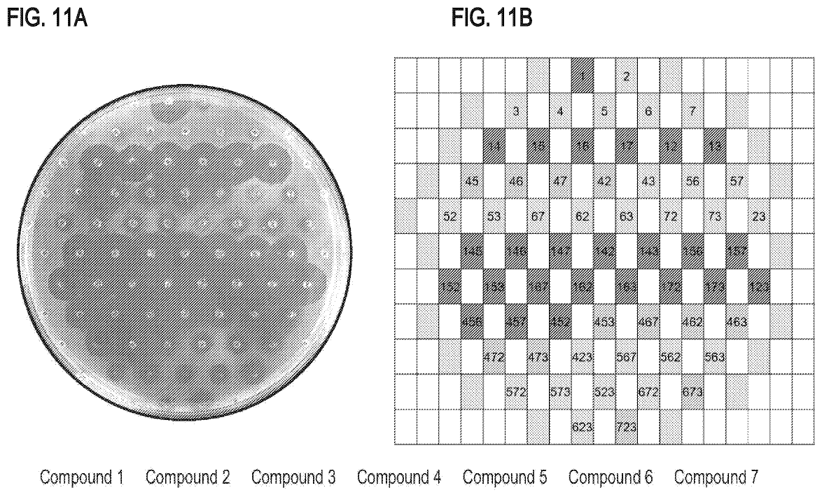

2. Pin array preparation: A pin array was loaded with one or more compounds according to the configurations shown in FIG. 11B. For instance, “142” denotes a combination of Compound 1, Compound 2, and Compound 3.

3. Treatment: After the cells reach the desired condition, the pin array loaded with ampicillin according to the configurations shown in FIG. 11B was coupled to an LB agar plate with E. coli culture. The LB agar plate was incubated at 37° C., and images of the plates were taken (FIG. 11A). The “hotness” indicates the level of inhibition in the zone of inhibition around each pin.

4. Results and analysis: The results are shown in FIGS. 11A and 11B, respectively. Well-defined zones of inhibition of different sizes can be observed in FIG. 11A for different combinations of compounds. These results demonstrate feasibility of the pin array device describe herein in studying the antibacterial effects of combinations of compounds.

We claim:

1. A pin array device for chemical or biological assays, comprising:
a plurality of first pins comprising a proximal end and a distal end, wherein the first pins comprise closed tips at the distal end of the pins;
a plate having a proximal surface and a distal surface;
wherein the plurality of first pins are integral to the plate and are on the distal surface of the plate, wherein the plurality of first pins form an array.

2. The device of claim 1, further comprising a second pin, wherein the second pin is capable of marking the orientation of the array.

3. The device of claim 2, wherein the second pin is outside of the array.

4. The device of any one of claims 1-3, wherein the plate is round and comprises a first rim on the distal surface of the plate.

5. The device of any one of claims 1-4, further comprising a protective dish, wherein the dish comprises a bottom plate and a second rim disposed on the bottom plate, wherein the bottom plate of the dish comprises a proximal surface and a distal surface.

6. The device of claim 5, wherein the plate is capable of fittingly engage the protective dish, and wherein the plate and the dish do not rotate relative to each other when a tight fit is formed.

7. The device of claim 5 or 6, wherein the length of the first pins is shorter than the distance between the distal surface of the plate and the proximal surface of the bottom plate when the plate and the protective dish fittingly engage each other, thereby preventing the closed tips of the first pins from touching the bottom plate.

8. The device of any one of claims 2-7, wherein the second pin is longer than the first pins, or the device further comprises a third pin on the proximal surface of the plate that is longer than the first pins.

9. The device of any one of claims 2-8, wherein the length of the second pin and/or the length of the third pin is about the same as the distance between the distal surface of the plate and the proximal surface of the bottom plate when the plate and the protective dish fittingly engage each other, thereby preventing the closed tips of the first pins from touching the bottom plate.

10. The device of any one of claims 1-9, wherein the first pins are pre-loaded with one or more compositions comprising an agent.

11. The device of claim 10, wherein
each of the first pins is loaded with the same composition comprising an agent at various amounts;
each of the first pins is loaded with a different composition comprising an agent;
some of the first pins are loaded with the same composition comprising an agent at various amounts; or
some of the first pins are loaded with a different composition comprising an agent.

12. The device of claim 11, wherein the composition comprises one or more antibiotic drugs.

13. The device of claims 11 or 12, wherein the one or more compositions comprising an agent are in the form of a powder, a solution, an emulsion, a film, or a gel.

14. The device of any one of claims 1-13, wherein the length of the first pins is about 2 mm to about 30 mm.

15. The device of any one of claims 1-14, wherein the length of the closed tips of the first pins is about 2 mm to about 30 mm.

16. The device of any one of claims 1-15, wherein the largest cross-section diameter of the closed tips of the first pins is about 0.5 mm to about 8 mm.

17. The device of any one of claim 1-16, wherein the distal end of the closed tips is concave.

18. The device of claim 17, wherein the depth of concaved distal end is about 1 μm to about 5 mm.

19. A kit for a chemical or biological assay comprising the device of any one of claims 1-18.

20. The kit of claim 19, wherein the kit further comprises one or more containers (e.g. petri dishes) capable of fittingly engage the device, and wherein the one or more containers and the device do not rotate relative to each other when a tight fit is formed.

21. The kit of claim 20, wherein the containers (e.g. petri dishes) contains one or more culture media, and optionally wherein the culture media is a solid phase media.

22. The kit of any one of claims 19-21, wherein the kit further comprises one or more compositions comprising an agent, and wherein the first pins and/or the second pin can be loaded with one or more compositions in the kit.

23. A method of performing a chemical or biological assay, comprising:
i) preparing a target cell culture (e.g. a bacteria) on a solid culture media in a dish;
ii) applying one or more compositions comprising an agent (e.g. an antibiotic drug) to tips of a plurality of first pins, wherein the first pins are integral to a plate that comprises a proximal surface and a distal surface, wherein the first pins are on the distal surface of the plate, and wherein the plurality of first pins form an array;
iii) stamping the tips of the plurality of first pins to the target cell culture, wherein the effects of the agents on the target cell culture are analyzed.

24. A method of performing a chemical or biological assay, comprising:
i) preparing a target cell culture (e.g. a bacteria) on a solid culture media in a dish;
ii) contacting the tips of a plurality of first pins pre-loaded with one or more compositions comprising an agent (e.g. an antibiotic drug) with the target cell culture, wherein the first pins are integral to a plate that comprises a proximal surface and a distal surface, wherein the first pins are on the distal surface of the plate, and wherein the plurality of first pins form an array,
iii) analyzing the effects of the agents on the target cell culture.

25. The method of claim 23 or 24, wherein the target cell culture comprises a bacteria and the compositions comprise one or more antibiotic drugs, and wherein the effects of the antibiotic drugs are analyzed by comparing sizes of inhibition zones around the tips of the first pins.

26. The method of claim 24 or 25, wherein the effects of the agents are analyzed while the plurality of first pins are in contact with the target cell culture.

27. The method of claim 24 or 25, wherein the effects of the agents are analyzed after the plurality of the first pins are separated from the target cell culture.

28. The method of any one of claims 24-27, wherein the plurality of the first pins are in contact with the target cell culture for at least about 30 minutes before the effects of the agents on the target cell culture are analyzed.
